(19) United States
(12) Patent Application Publication
(10) Pub. No.: US 2010/0190982 A1
(43) Pub. Date: Jul. 29, 2010

(54) PROCESS FOR THE PREPARATION OF LAMIVUDINE FORM I

(76) Inventors: Janardhana Rao Vascuri, Hyderabad (IN); Ravinder Reddy Vennapureddy, Hyderabad (IN); Shankar Rama, Hyderabad (IN); Ramesh Dandala, Hyderabad (IN); Sivakumaran Meenakshisunderam, Hyderabad (IN)

Correspondence Address: Jay R. Akhave, 2058 N Mills Ave. #612, Claremont, CA, 91711, US

(21) Appl. No.: 12/733,690

(22) PCT Filed: Sep. 1, 2008
(86) PCT No.: PCT/IB2008/002276

§ 371 Date: Mar. 15, 2010

(30) Foreign Application Priority Data
Sep. 17, 2007 (IN) 2071/CHE/2007

(51) Int. Cl. C07D 239/47

(52) U.S. Cl. 544/317

(57) ABSTRACT

The present invention provides a process for preparing a stable crystalline solid of Lamivudine polymorphic Form I, which does not change to Form II during storage and pharmaceutical unit operations.

### FIELD OF THE INVENTION

The present invention relates to the stable lamivudine polymorphic Form I.

The present invention also relates to a process for the preparation of lamivudine of Formula I,

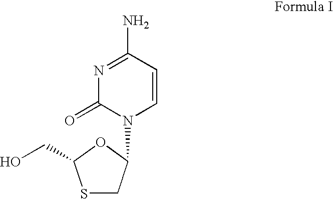

in polymorphic Form I having high stability.

### BACKGROUND OF THE INVENTION

Lamivudine of Formula I is an antiviral drug presently marketed by GlaxoSmithkline and is available as “EPIVIR”, indicated for the treatment against retroviruses such as Human immuno deficiency virus (HIV), Hepatitis B virus (HBV) and Human T-Lymphotrophic virus (HTLV).

WO 91/17159 A1 describes the preparation of Lamivudine (3TC), its antiviral activity and its use in pharmaceutical product. 3TC is described and prepared in WO 91/17159 Al as a freeze dried powder.

U.S. Pat. No. 5,905,082 discloses the existence of two polymorphic forms of Lamivudine viz., needle-shaped crystals (Form I) and bipyramidal crystals (Form II). It has also been established (J. Chem. Soc. Perkin Trans 2, 1997, 2653) that Form I is a hydrate (having one molecule of water to every five molecules of Lamivudine). It is stated that when Lamivudine is crystallized from aqueous solution or methanol, needle-shaped crystals (Form I) are obtained and when it is crystallized from non-aqueous solvents substantially bipyramidal crystals (Form II) are obtained.

The two polymorphic forms have been distinguished by their XRD, DSC, IR and melting range. Form II has a melting point of 177-178° C. and its IR spectrum exhibits strong absorption bands at ˜920 and ˜850 cm⁻¹. Further, Form I shows a characteristic band at 1110 cm⁻¹, which is absent in Form II. Similarly the ˜920 and ˜850 cm⁻¹ bands are absent in Form I. Form I has a melting point of 124-127° C.

Further, U.S. Pat. No. 5,905,082 states that Form II is the more stable polymorphic form and used for the preparation of pharmaceutical products. It also discloses that Form I crystals are less stable and in certain pharmaceutical unit operations such as milling/grinding may cause conversion of Form I to Form II, which is an undesirable characteristic for manufacture of solid dosage forms and thus is not favored for the pharmaceutical formulation. In U.S. Pat. No. 5,905,082, it is suggested that Form II crystals can be obtained by grinding or milling Form I. Also Form II has been prepared by slurrying Lamivudine Form I in solvents such as Methylated spirit. All these indicate the instability of Form I known in prior art. However, we have prepared stable Lamivudine Form I crystals, which do not convert into Form II, during the preparation of solid pharmaceutical dosage forms and during storage.

### OBJECTIVE

The main object of the present invention is to provide stable lamivudine polymorphic Form I.

Another object of the present invention is to prepare lamivudine polymorphic Form I, which is stable and does not convert to other polymorphic forms.

### SUMMARY OF THE INVENTION

The present invention relates to the stable Lamivudine polymorphic Form I of Formula I,

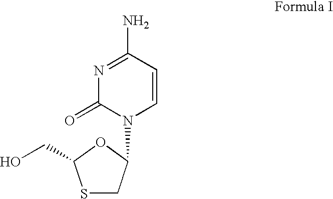

The present invention also relates to a process for the preparation of Lamivudine polymorphic Form I having high stability, which comprises:
a) dissolving Lamivudine in a mixture of ethanol and water at 45-55° C.;
b) optionally filtering the solution through hyflo at 45-55° C. to remove undissolved particles if any;
c) removing ethanol under reduced pressure below 42° C. to obtain product as a solid residue;
d) precipitating the product by addition of ethyl acetate/methyl isobutyl ketone to obtain a free flowing solid; and
e) filtering the product and drying the wet material below 40° C. under reduced pressure till the water content is ≦1.8% w/w.

The present invention also relates to a process for the preparation of Lamivudine polymorphic Form I having high stability, which comprises:
a) treating Lamivudine salicylate monohydrate with an organic base in an organic solvent at 20-25° C.; and
b) isolating the Lamivudine polymorphic Form I in stable form.

The present invention also relates to a process for the preparation of Lamivudine polymorphic Form I having high stability, which comprises:
a) slurrying Lamivudine in a mixture of ethyl acetate and water; and
b) isolating the Lamivudine Form I in stable form.

### BRIEF DESCRIPTION OF THE DRAWINGS

FIG. 1—IR spectrum of Lamivudine polymorphic Form I having high stability.

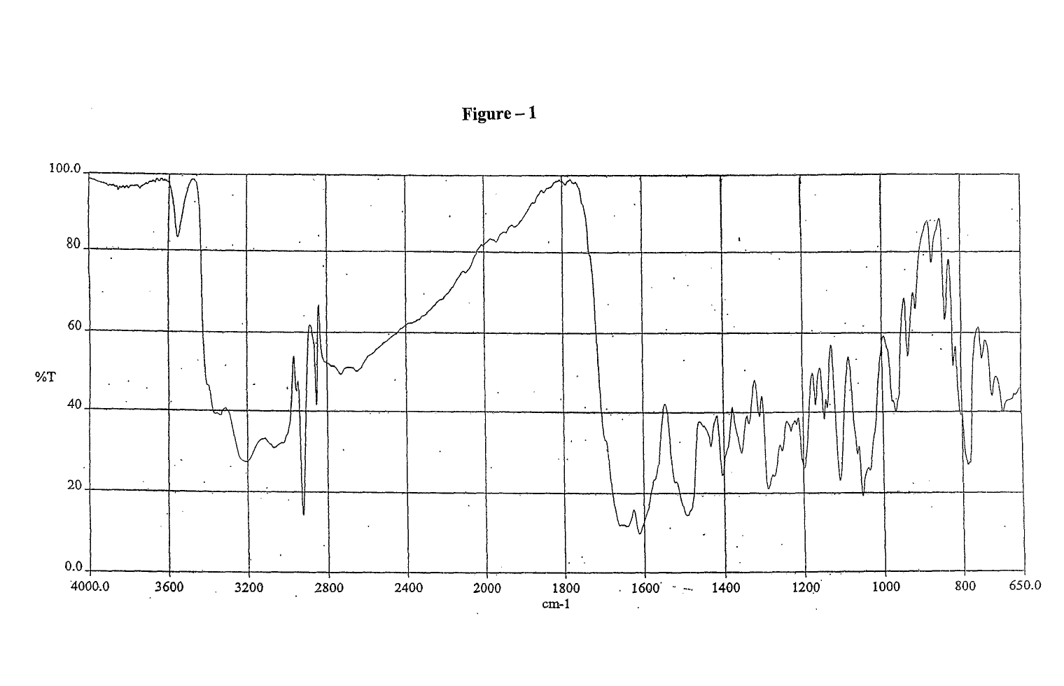

FIG. 2—Raman spectrum of Lamivudine polymorphic Form I having high stability.

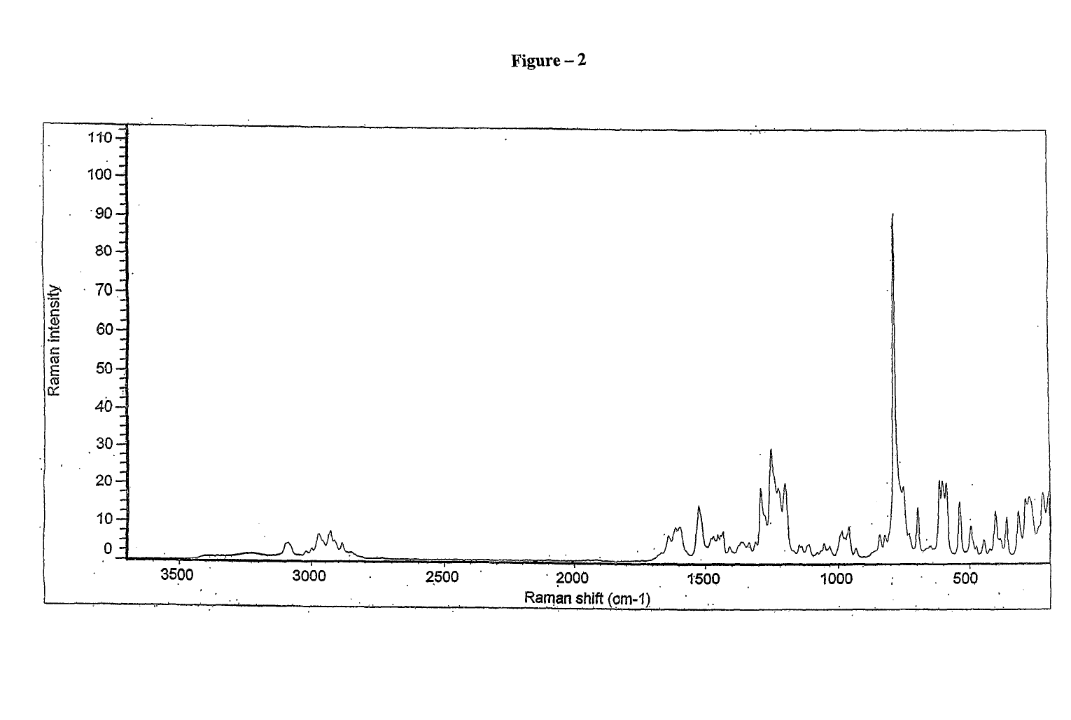

FIG. 3—XRD of Lamivudine polymorphic Form I having high stability.

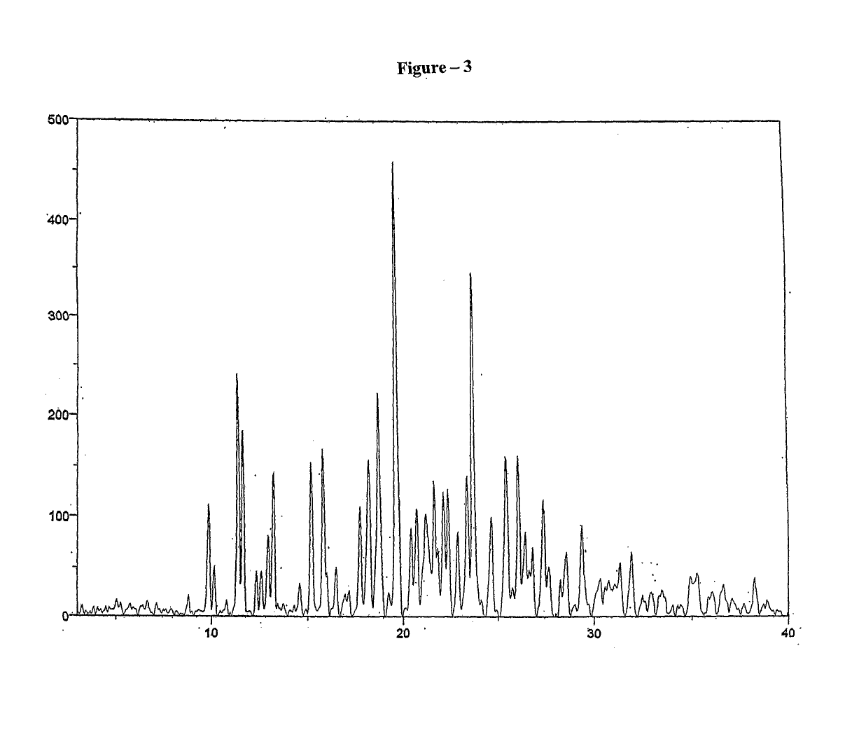

FIG. 4—DSC of Lamivudine polymorphic Form I having high stability.

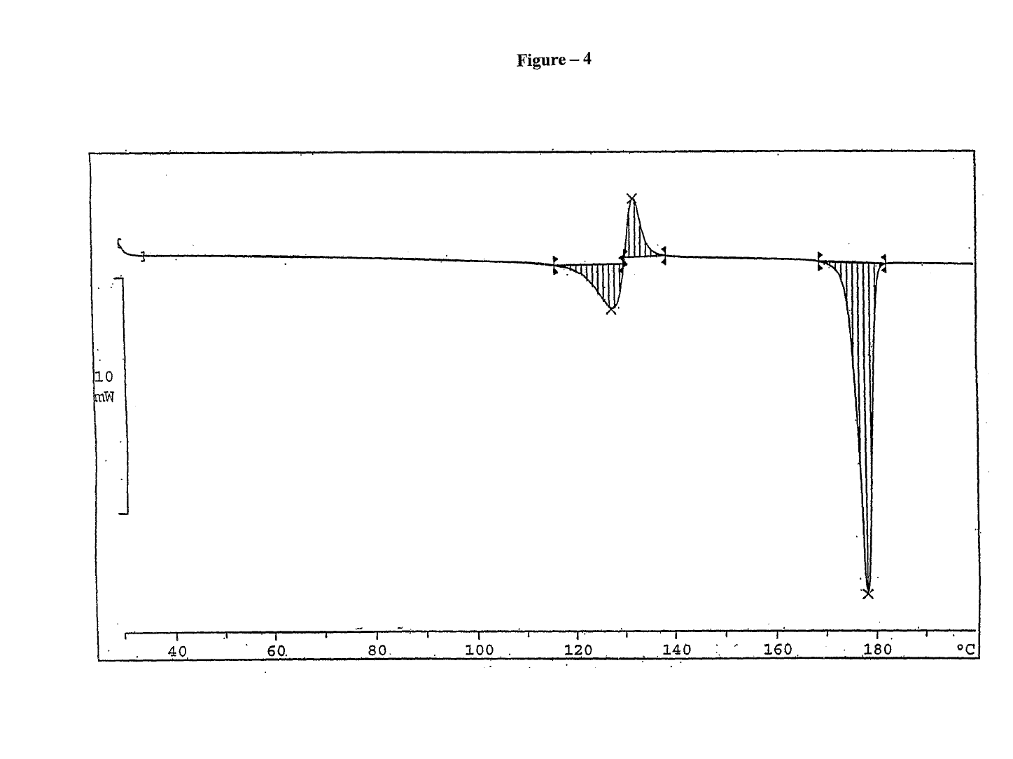

### DETAILED DESCRIPTION OF THE INVENTION

It is necessary to provide a single polymorphic form for certain pharmaceutical unit operations, as a mixture of polymorphic forms gives undesirable characteristics of solid dosage forms. These can result in inconsistent bioavailability, difficulties in powder processing and tablet formation. Also during pharmaceutical operations the polymorph should preferably remain unchanged in order to get consistent bioavailability etc.

The present invention provides a stable Lamivudine polymorphic Form I having no or little tendency to convert to any other polymorphic Form of Lamivudine.

In the case of Lamivudine polymorphic Form I prepared by crystallisation from water as per the prior art procedure (U.S. Pat. No. 5,905,082, Example 1), we observed that the Form I crystals get partially converted into Form II on storage or on drying at 80° C. Also partial conversion to Form II was observed when those materials were milled/grinded. Therefore our endeavour was to prepare Lamivudine polymorph Form I, which is storage stable and remains unchanged during preparation of solid doses form. Surprisingly, it was found that the Lamivudine Form I crystals prepared by the process of instant invention do not convert to Form II on drying at 80° C. (under reduced pressure). Data has been generated up to 72 h drying at 80° C. (under reduced pressure) and no polymorphic change has been observed. Also the Form I produced by instant invention was subjected to stability testing at 60° C. and neither chemical degradation nor polymorphic change was observed during two months of stability study. Tablets have been prepared using the Form I crystals obtained by present invention and polymorphic purity was evaluated for the blend, uncoated and coated tablets and no conversion to Form II was observed. Form I crystals of present invention have also been grinded neat for 5-15 min at 20-30° C. and no change in polymorphic form has been observed.

Lamivudine, used in the preparation of stable Lamivudine Form I, is amorphous Lamivudine, Lamivudine Form II. or a mixture of Form I or Form II.

Stable Lamivudine Form I is prepared by crystallization of Lamivudine by dissolving in aqueous alcoholic solvent, preferably in 5-30% aqueous alcohol, more preferably in 15-20% aqueous alcohol. Both aqueous methanol or ethanol can be used, however, solvent most preferred for dissolution is 15-20% aqueous ethanol at 35-60° C., preferably at 45-55° C. The solution is filtered through celite to obtain a clear filtrate, which is free from undissolved Lamivudine or extraneous matter. The solution is concentrated under reduced pressure below 42° C. When the temperature is above 55° C. during the concentration, either a mixture of polymorphic Form I and Form II or only Form II is obtained. The Lamivudine polymorph Form I obtained from aqueous methanol using the process of instant invention has similar stability profile, however, the product was found to contain 1.0-2.0% of residual methanol, which could not be removed by drying. However, no residual ethanol in high content was observed when Form I is prepared from ethanol.

In another aspect of the invention, Lamivudine polymorph Form I is obtained by slurring of Lamivudine in aqueous ethyl acetate and water at 20-30° C. Water content in ethyl acetate can be from 2-5% w/w.

Another aspect of the invention provides a process for the preparation of Lamivudine Form I from Lamivudine salicylate monohydrate, which involves treating Lamivudine salicylate monohydrate with an organic base preferably triethylamine in an organic solvent to neutralize and isolate Lamivudine Form I by filtration of the slurry. The solvents selected for this transformation are ethyl acetate, methylisobutyl ketone, acetone etc. and most preferably ethyl acetate.

In the present invention, the Lamivudine polymorphic Form I obtained is 100% pure. Other polymorphic form is always below the detectable limit (<1.0%) and the polymorph does not change into other polymorphic form upon drying (up to 80° C.), which is a desirable characteristic for solid dosage preparation.

The stable Lamivudine Form I product, which is needle shaped crystals, shows a DSC profile similar to that reported for Form I with an onset temperature 121.4-129° C. IR spectrum of polymorphic Form I obtained by the present invention exhibits a strong absorption band at about 1109 cm⁻¹, and shows no bands at ˜920 and ˜850 cm^{-1,} which correspond to polymorphic Form II. Powder XRD pattern of polymorphic Form I of the present invention shows characteristic peaks at 20 values of 15.46°, 18.9° and shows no peaks at 2θ values of 14.36°, 17.6° and also no prominent peaks at 20.69°, 21.6°, 26.56°, which corresponds to polymorphic Form II.

A Fourier Transform Raman spectroscopy method was also used for mathematically determining the polymorphic ratio in pharmaceutical composition. Raman spectrum of Lamivudine polymorphic Form I shows characteristic peaks in the range of 707.83-686.61 cm⁻¹ and 333.16-300.83 cm⁻¹. Raman Spectrum of Lamivudine polymorphic Form II shows characteristic peaks in the range of 1188.02-1176.44 cm⁻¹, 466.77-457.13 cm⁻¹ and a prominent characteristic peak in the range of 804.25-790.75 cm⁻¹.

Quantification of Lamivudine Form II in Form I by FT-Raman Spectrum during Pharmaceutical Dosage Form Preparation:

| Product | Form I (%) | Form II (%) |
|---|---|---|
| Final blend | ≅99.59 | ≅0.41 |
| Final blend | ≅100 | Not detected |
| Uncoated tablet | ≅99.68 | ≅0.32 |
| Uncoated tablet | ≅100 | Not detected |
| Coated tablet | ≅100 | Not detected |
| Coated tablet | ≅99.95 | ≅0.05 |

The invention is illustrated with the following examples, which are provided by way of illustration only and should not be construed to limit the scope of the invention.

### Example-1

Lamivudine (polymorphic Form II, 100 g) was dissolved in a mixture of ethanol (680 ml) and water (.120 ml) at 45-52° C. Activated carbon (3 g) was added to the solution and stirred for 10 min. The carbon was removed by filtration through hyflo and washed the residue with 20% v/v aqueous ethanol (100 ml). Ethanol-water mixture (˜680 ml) was distilled out from clear filtrate under reduced pressure (=100 mm Hg) below 42° C. to a pot volume of ˜130 ml. Ethyl acetate (500 ml) was added to the pot residue at 28-32° C. in a single lot and stirred for 2 h to complete the precipitation of the product. The product was collected by filtration, washed with ethyl acetate (200 ml) and dried under reduced pressure (=50 mm Hg) at 40-50° C. till the water content was ≦1.8% w/w to yield Lamivudine polymorphic Form I (88 g) mp. 124-129° C.

### Example-2

4-Amino-1-[(2R,5 S)-2-(hydroxymethyl)-1,3 -oxathiolan-5-yl]-(1H)-pyrimidin-2-one monosalicylate monohydrate (Lamivudine salicylate, 100 g) was added slowly over 30 min to a mixture of triethylamine (55 g) and ethyl acetate (650 ml), under stirring at 25-30° C. The resulting product slurry was stirred for 2 h at 25-30° C. The product was filtered and washed with ethyl acetate (100 ml) at 25-30° C. The solid obtained was dried under reduced pressure at 50° C. to yield the polymorphic Form I of Lamivudine (58 g), mp 122-124° C.

The above obtained Lamivudine Form I can be further recrystallized using the procedure described in example 1.

### Example-3

Lamivudine polymorphic Form II (15 g) was dissolved in a mixture of methanol (120 ml) and water (18 ml) at 45-52° C. Activated carbon was added to the solution and stirred for 10 min at 45-52° C. The carbon was removed by filtration through hyflo and washed the residue with 20% v/v aqueous methanol (30 ml). Methanol-water mixture was distilled under reduced pressure (˜200 mm Hg) below 45° C. to a pot volume of 20 ml. Ethyl acetate (75 ml) was added to the residue in a single lot at 28-32° C. and stirred for 1 h at 28-32° C. to complete the precipitation of the product. The product was filtered, washed with ethyl acetate (30 ml) and dried under reduced pressure (˜50 mm Hg) till the water content was ≦1.8% w/w at 40-50° C. to yield Lamivudine polymorphic Form I (13.2 g), mp 123-128° C. Residual methanol: 1.9%w/w

### Example-4

Lamivudine (polymorphic Form II, 50 g) was dissolved in a mixture of ethanol (340 ml) and water (60 ml) at 45-52° C. Activated carbon (1.5 g) was added to the solution and stirred for 10 min. The carbon was removed by filtration through hyflo and washed the residue with 20% v/v aqueous ethanol (60 ml). Ethanol-water mixture (˜350 ml) was distilled out from clear filtrate under reduced pressure (˜100 mm Hg) below 42° C. to a pot volume of ˜60 ml. Methylisobutyl ketone (225 ml) was added to the pot residue at 28-32° C. in a single lot and stirred for 2 h to complete the precipitation of the product. The product was collected by filtration, washed with Methylisobutyl ketone (100 ml) and dried under reduced pressure (˜50 mm Hg) at 40-50° C. till the water content was ≦1.8% w/w to yield Lamivudine polymorphic Form I (45 g), mp. 124.5-129° C.

### Example-5

4-Amino-1-[(2R,5S)-2-(hydroxymethyl)-1,3-oxathiolan-5-yl]-(1H)-pyrimidin-2-one monosalicylate monohydrate (Lamivudine salicylate, 100 g) was added slowly over 30 min to a mixture of triethylamine (55 g) and Methylisobutyl ketone (700 ml), under stirring at 25-30° C. The resulting product slurry was stirred for 2 h at 25-30° C. The product was filtered and washed with Methylisobutyl ketone (100 ml) at 25-30° C. The solid obtained was dried under reduced pressure at 50° C. to yield the polymorphic Form I of Lamivudine (59 g), mp 123-126.5° C.

The above obtained Lamivudine Form I can be further recrystallized using the procedure described in example 4.

### Example-6

Lamivudine (mixture of Form I and Form II, 30 g) was added to a mixture of ethyl acetate (210 ml) and purified water (5 ml) containing triethylamine (0.2 g) at 20-30° C. The slurry was stirred over night at 20-30° C. The product was collected by filtration washed with ethyl acetate (60 ml) and dried under reduced pressure (˜50mm Hg) at 40-45° C. to yield Lamivudine polymorphic Form I (26.2 g), nip 128-130° C.

We claim:

1) A process for the preparation of Lamivudine of Formula I 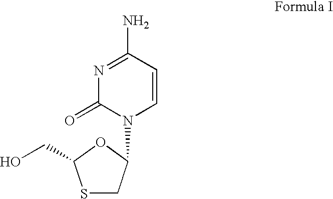
in polymorphic Form I having high stability, which comprises:
a) dissolving Lamivudine in aqueous ethanol at 45-55° C.;
b) optionally filtering the solution through hyflo at 45-55° C. to remove undissolved particles if any;
c) removing ethanol under reduced pressure below 42° C. to obtain product as a solid residue;
d) precipitating the product by addition of ethyl acetate/methyl isobutyl ketone to obtain a free flowing solid; and
e) filtering the product and drying the wet material till the water content is ≦1.8% w/w.

2) The process according to claim 1, wherein the drying in step
(e) is carried out below 40° C. under reduced pressure.

3) A process for the preparation of Lamivudine of Formula I. 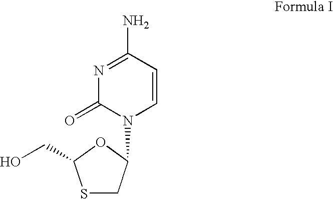
in polymorphic Form I having high stability, which comprises:
a) treating Lamivudine salicylate monohydrate with an organic base in an organic solvent at 20-25° C.; and
b) isolating the Lamivudine polymorphic Form I in stable form.

4) The process according to claim 3, wherein the organic base is triethylamine.

5) The process according to claim 3, wherein the organic solvent is selected from ethyl acetate, methylisobutyl ketone, acetone.

6) The process according to claim 5, wherein the organic solvent is ethyl acetate.

7) A process for the preparation of Lamivudine of Formula I 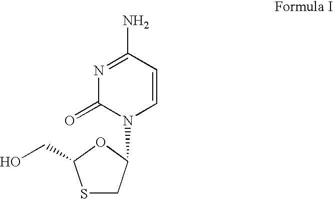
in polymorphic Form I having high stability, which comprises:
a) slurrying Lamivudine in aqueous ethyl acetate; and
b) isolating the Lamivudine polymorphic Form I in stable form.

8) Lamivudine polymorphic Form I having high stability.

9) The stable Lamivudine polymorphic Form I according to claim 7, wherein the Lamivudine has no detectable quantity of any other polymorphic Form during storage or drying at higher temperatures or during pharmaceutical dosage form preparations.

10) The stable Lamivudine polymorphic Form I according to claim 1, wherein the Lamivudine has no detectable quantity of any other polymorphic Form during storage or drying at higher temperatures or during pharmaceutical dosage form preparations.

11) The stable Lamivudine polymorphic Form I according to claim 3, wherein the Lamivudine has no detectable quantity of any other polymorphic Form during storage or drying at higher temperatures or during pharmaceutical dosage form preparations.
